SEQUENCE LISTING (13 sequences)

SEQ ID NO: 1 - DNA, 1413 bp, Oryza sativa
atggacctcgccgccgccgccgtggccgtctccttcccacgccctgctcccccgccgcgccgctgcgccccacgccgccaccgccgcgccctcgctccgcgcgcggcctcctcctccccctccccctccacggcggtggcggcgcccgtctacgcccccacgccgcgggaccgggccctgcggacgccgcacagcgggtatcactatgacggcacggcgaggcccttctttgagggatggtacttcaaggtgtccattcccgagtgcaggcagagcttctgcttcatgtactctgtcgagaacccgttgttccgagatgggatgagtgatctggatcgggtcatacatggttcgcgcttcactggcgtcggggcgcagattcttggcgccgatgataagtacatatgccagttcaccgagaaatccaataacttttggggaagtaggcatgaactaatgctcggaaacactttcattcccaataatggttcaacacccccagaaggggaggttccccctcaggaattttctagtcgagttttggaaggcttccaagtgacaccgatttggcatcaaggctttatacgtgatgatggaaggtcaaagtatgtgccaaatgtccaaacagctaggtgggagtacagcactcgaccagtatatggctggggtgatgtcacgtcaaagcagaaatcaactgctggttggcttgctgcttttcctttctttgaacctcattggcaaatatgcatggctggtggcttatccacaggatggattgaatgggacggagagcggtttgaatttgaaaatgctccttcttattcagaaaagaactggggtgcaggttttccgaggaagtggtattgggtccagtgcaatgtcttctcaggcgcatctggtgaagttgcattaacggctgctggcggattaaggaaaattggattgggcgaaacctatgaaagtccttcactgattggaattcattatgagggaaaattctatgaatttgtgccttggaccgggacagtgagctgggacattgccccttggggtcactggaagttgtccggcgagaacaaaaatcatctggttgaaatagaagcaaccacaaaagaaccaggcactgctttgcgagctccaaccatggaggctggactagtgccagcatgcaaagacacctgctatggtgatctgaggctgcaaatgtgggaaaagagaaatgatggtggcaagggaaagatgatactcgacgcaacaagcaacatggcggcactagaagttggtggaggcccttggttcaatggttggaaaggcacgactgtttcaaacgagattgtgaacaacgttgttggtacccaggtcgatgtggagagcctcttccctatcccatttctcaagccccctggtctgtag

SEQ ID NO: 2 - protein, 470 aa, Oryza sativa
MDLAAAAVAVSFPRPAPPPRRCAPRRHRRALAPRAASSSPSPSTAVAAPVYAPTPRDRALRTPHSGYHYDGTARPFFEGWYFKVSIPECRQSFCFMYSVENPLFRDGMSDLDRVIHGSRFTGVGAQILGADDKYICQFTEKSNNFWGSRHELMLGNTFIPNNGSTPPEGEVPPQEFSSRVLEGFQVTPIWHQGFIRDDGRSKYVPNVQTARWEYSTRPVYGWGDVTSKQKSTAGWLAAFPFFEPHWQICMAGGLSTGWIEWDGERFEFENAPSYSEKNWGAGFPRKWYWVQCNVFSGASGEVALTAAGGLRKIGLGETYESPSLIGIHYEGKFYEFVPWTGTVSWDIAPWGHWKLSGENKNHLVEIEATTKEPGTALRAPTMEAGLVPACKDTCYGDLRLQMWEKRNDGGKGKMILDATSNMAALEVGGGPWFNGWKGTTVSNEIVNNVVGTQVDVESLFPIPFLKPPGL

SEQ ID NO: 3 - DNA, 1413 bp, Artificial Sequence
  Variant VTE1 coding sequence
atggacctggctgctgctgctgtggctgtgtccttcccaaggccagctccaccaccaaggaggtgcgccccgaggaggcacaggagggccctggccccgagggccgcttcttccagcccgtccccgtccaccgccgtggccgccccggtgtacgccccgaccccgagggacagggccctgaggaccccacactccggataccactacgacggaaccgctaggccgttcttcgagggctggtacttcaaggtgtccatcccggagtgcaggcagtccttctgcttcatgtactccgtggagaacccgctgttcagggacggcatgtccgacctggacagggtgatccacggctccaggttcaccggagtgggcgcccagatcctgggagccgacgacaagtacatctgccagttcaccgagaagtccaacaacttctggggctccaggcacgagctgatgctgggaaacaccttcatcccaaacaacggatccaccccaccagagggagaggtgccaccacaggagttctcctccagggtgctggagggattccaggtgaccccgatctggcaccagggcttcatcagggacgacggcaggtccaagtacgtgccgaacgtgcagaccgcccgctgggagtactccaccaggccagtgtacggatggggagacgtgacctccaagcagaagtccaccgctggatggctggctgctttcccgttcttcgagccacactggcagatctgcatggctggaggactgtccaccggatggatcgagtgggacggcgagaggttcgagttcgagaacgccccgtcctactccgagaagaactggggcgccggcttcccgaggaagtggtactgggtgcagtgcaacgtgttctccggagcttccggagaggtggctctgaccgctgctggaggactgaggaagatcggactgggcgagacctacgagtccccgtccctgatcggcatccactacgagggcaagttctacgagttcgtgccgtggaccggaaccgtgtcctgggacatcgctccgtggggacactggaagctgtccggcgagaacaagaaccacctggtggagatcgaggctaccaccaaggagccaggaaccgctctgagggccccgactatggaggctggactggtgccagcctgcaaggacacctgctacggcgacctgaggctgcagatgtgggagaagaggaacgacggcggcaagggaaagatgatcctggacgctacctccaacatggctgctctggaagtgggaggcggcccgtggttcaacggatggaagggaaccaccgtgtccaacgagatcgtgaacaacgtggtgggcacccaggtggacgtggagtccctgttcccgatcccgttcctgaagccgccgggcctgtga

SEQ ID NO: 4 - DNA, 20 bp, Artificial Sequence
  Primer  RT-OsVTE1
agggcctattcatctctacc

SEQ ID NO: 5 - DNA, 24 bp, Artificial Sequence
  Primer RT-OsVTE2
ggtgtccattcccgagtgcaggca

SEQ ID NO: 6 - DNA, 20 bp, Artificial Sequence
  Primer Rtime1
tgcaatgtcttctcaggcgc

SEQ ID NO: 7 - DNA, 20 bp, Artificial Sequence
  Primer Rtime2
gcttctatttcaaccagatg

SEQ ID NO: 8 - DNA, 28 bp, Artificial Sequence
  PCR primer
ccaagcttgcacgaccataggcgtgggt

SEQ ID NO: 9 - DNA, 28 bp, Artificial Sequence
  PCR primer
gctctagagctgatgctgcgggcgggca

SEQ ID NO: 10 - DNA, 29 bp, Artificial Sequence
  RT-PCR primer
cggggtaccagggcctattcatctctacc

SEQ ID NO: 11 - DNA, 29 bp, Artificial Sequence
  RT-PCR primer
cgcggatccagcatcagcatggacctcgc

SEQ ID NO: 12 - DNA, 33 bp, Artificial Sequence
  Primer RNAiF
tgctctagagagctccagttcaccgagaaatcc

SEQ ID NO: 13 - DNA, 33 bp, Artificial Sequence
  Primer RNAiR
accgtcgacgagctcagatgcgcctgagaagac

(19) United States
(12) Patent Application Publication
(10) Pub. No.: US 2012/0272352 A1
(43) Pub. Date: Oct. 25, 2012

(54) Genes Conferring Drought and Salt Tolerance and Uses Thereof

(75) Inventors: Shouyi Chen, Beijing (CN); Jinsong Zhang, Beijing (CN); Shouqiang Ouyang, Beijing (CN); Sijie He, Beijing (CN); Wanke Zhang, Beijing (CN); Biao Ma, Beijing (CN); Qing Lin, Beijing (CN)

(73) Assignees: Syngenta Participations AG, Basel (CN); Institute of Genetics and Developmental Biology, Chinese Academy of Sciences, Beijing (CN)

(21) Appl. No.: 13/504,846

(22) PCT Filed: Oct. 12, 2010
(86) PCT No.: PCT/CN2010/077688

§ 371 Date: Jul. 9, 2012

(30) Foreign Application Priority Data
Oct. 30, 2009 (CN) PCT/CN2009/074722

(51) Int. Cl. A01H 1/06; A01H 1/02

(52) U.S. Cl. 800/260; 800/278

(57) ABSTRACT

Compositions and methods for conferring drought and salt tolerance to plants using tocopherol cyclase (TC) genes, including polynucleotides, polypeptides, vectors, cells and plants.

### TECHNICAL FIELD OF THE INVENTION

The invention relates generally to compositions and methods for conferring drought and salt tolerance to plants using tocopherol cyclase (TC) genes. The aforementioned compositions include polynucleotides, polypeptides, vectors and host cells. The present invention also relates to plants transformed by the aforementioned compositions and methods.

### BACKGROUND OF THE INVENTION

Over the course of their lifetime, plants are exposed to ever-changing environments and various biotic and abiotic stresses. High salinity and drought are the major abiotic stresses, and both reduce plant growth and agricultural productivity. An important mediator of these stresses is the accelerated generation and/or accumulation of reactive oxygen species (ROS), including hydrogen peroxide, hydroxyl radicals and superoxide anion, which damage the cellular components and may even cause death (Inze et al., 1995; Allen et al., 1995; Bolwell et al., 1997; Lamb et al., 1997; Noctor et al., 1998; Orozco-Cardenas et al., 1999; Karpinski et al., 1999).

To cope with oxidative stress, plants have developed an antioxidative system consisting of both enzymatic and non-enzymatic components, the latter of which includes tocopherol and tocotrienols—collectively known as vitamin E (Dat et al., 2000; Alscher and Heath, 2002). Vitamin E performs numerous critical functions, including quenching and scavenging various reactive oxygen species and free radicals and protecting polyunsaturated fatty acids from lipid peroxidation. For example, experiments performed in an Arabidopsis tocopherol cyclase mutant (vte 1) and homogentisate phytyltransferase (HPT) mutant (vte2) showed that, under high light intensity combined with low temperature, tocopherols protect against peroxidative damage in leaf disks (Havaux et al., 2005).

Tocopherol and tocotrienols are amphiphilic lipids synthesized exclusively by photosynthetic organisms (Fryer, 1992; Bramley et al., 2000; Wang and Quinn, 2000; Munne-Bosch and Alegre, 2002). Tocopherol and tocotrienols are composed of a polar chromanol head and a lipophilic isoprenoid tail derived from homogentisate and phytyl diphosphate respectively. In membrane lipid bilayers, the polar chromanol head is exposed to the surface of membrane and the lipophilic isoprenoid tail combines with lipide. There are four forms of tocopherol (α-, β-, γ- and δ-), and they differ from one another only in the number and position of methyl substituents attached to the chromanol ring.

The tocopherol synthesis pathway has been studied over the past three decades, and tocopherol cyclase (TC) is one of the enzymes in this pathway (See FIG. 1; See also Soll et al., 1980, 1985; Lichtenthaler et al., 1981; d'Harlingue and Camara, 1985; Norris et al., 1995, 1998; Arango and Heise, 1998; Shintani and DellaPenna, 1998; Collakova and DellaPenna, 2001; Schledz et al., 2001; Porfirova et al., 2002; Savidge et al., 2002; Cheng et al., 2003; Sattler et al., 2003). TC activity is evolutionarily conserved between plants and cyanobacteria (Sattler et al., 2003), and the tocopherol cyclase AtVTE1 is a major limiting factor of tocopherol synthesis in Arabidopsis thaliana leaves (Kanwischer et al., 2005).

In most instances, α-tocopherol is the predominant form of tocopherol in leaves and γ-tocopherol is most abundant form of tocopherol in seeds (Grusak and Della Penna, 1999). In transgenic tobacco plants constitutively silenced for homogentisate phytyltransferase (HPT) and γ-tocopherolmethyltransferase (γ-TMT) activity, both tocopherols have been shown to play a role in abiotic stress responses, though these roles differ (Abbasi et al., 2007). For example, γ-tocopherol was more potent than α-tocopherol in conferring desiccation tolerance in vivo, though γ-tocopherol could not substitute for α-tocopherol in surviving salt stress, although markers for oxidative stress were decreased in γ-TMT transgenic plants compared to wild type.

However, in contrast to what one might expect, expression levels of the various pathway enzymes are not necessarily affected by abiotic stresses. For example, in contrast to HPPD and HPT1, the expression level of AtVTE1 was not significantly altered during stress in Arabidopsis (Collakova et al., 2003b). Thus, there is no direct correlation between levels of these enzymes and abiotic stress responses. Accordingly, finding ways to address prevalent abiotic stresses such as high salt and drought are needed, especially in important food staples like rice and corn.

### BRIEF SUMMARY OF THE INVENTION

The present invention relates to isolated TC polynucleotides, polypeptides, vectors and host cells expressing isolated TC polynucleotides capable of conferring increased drought and salt tolerance to plants. The related polynucleotides, polypeptides, vectors and cells of the present invention are also capable of imparting specific traits to plants, such as decreased chlorophyll loss in response to salt stress, reduced production of H₂O₂ and other reactive oxygen species (ROS), and increased production of γ-tocopherol.

The isolated TC polynucleotides provided herein include nucleic acids comprising (a) a nucleotide sequence of SEQ ID NO: 1; (b) a nucleotide sequence of SEQ ID NO: 3; (c) a nucleotide sequence at least 70% identical to (a) or (b); (c) those that specifically hybridize to the complement of (a) or (b) under stringent hybridization conditions; (d) an open reading frame encoding a protein comprising a polypeptide sequence of SEQ ID NO: 2; (e) an open reading frame encoding a protein comprising a polypeptide sequence at least 70% identical to SEQ ID NO: 2; and (f) a nucleotide sequence that is the complement of any one of (a)-(f).

The isolated TC polypeptides provided herein include (a) an amino acid sequence of SEQ ID NO: 2 and (b) an amino acid sequence at least 70% identical to (a).

The host cells provided herein include those comprising the isolated polynucleotides and vectors of the present invention. The host cell can be from an animal, plant, or microorganism, such as E. coli. Plant cells are particularly contemplated. The host cell can be isolated, excised, or cultivated. The host cell may also be part of a plant.

The present invention further relates to a plant or a part of a plant that comprises a host cell of the present invention. Rice and corn are particularly contemplated. The present invention also relates to the transgenic seeds of the plants.

The present invention further relates to a method for producing a plant comprising regenerating a transgenic plant from a host cell of the present invention, or hybridizing a transgenic plant of the present invention to another non-transgenic plant. Plants produced by these methods are also encompassed by the present invention, and rice is particularly contemplated.

The present invention further relates to methods of altering a trait in a plant or part of a plant using the isolated polynucleotides, polypeptides, constructs and vectors of the present invention. These traits include conferring increased salt tolerance, increased drought tolerance, decreased chlorophyll loss in response to salt stress, decreased ROS production, and increased γ-tocopherol production. In one embodiment, these traits are increased or improved by increasing the expression of TC nucleic acids or proteins of the invention, such as SEQ ID NOs: 1-3.

The present invention further relates to the use of the isolated polynucleotides, polypeptides, constructs and vectors of the present invention to alter plant traits, e.g., salt tolerance, drought tolerance, decreased chlorophyll loss in response to salt stress, and increased γ-tocopherol production. In one embodiment, these traits are increased or improved by increasing the expression of TC nucleic acids or proteins of the invention, such as SEQ ID NOs: 1-3.

### BRIEF SUMMARY OF THE SEVERAL VIEWS OF THE DRAWINGS

FIG. 1 illustrates the tocopherol biosynthetic pathway. Dashed arrows represent multiple steps. Enzymes are indicated by circled numbers: 1, HGA phytyltransferase (EMT); 2, p-hydroxyphenyl pyruvate dioxygenase (HPPD); 3, HGA dioxygenase; 4, geranylgeranyl diphosphate reductase (GGDR); 5, geranylgeranyl diphosphate synthase (GGPS); 6,1-deoxy-D-xylulose-5-phosphate synthase (DXPS); 7,2-methyl-6-phytyl-1,4-benzoquinolmethyltransferase (MPBQ); 8, tocopherol cyclase (TC); and 9, γ-tocopherol methyltransferase (γ-TMT) (Collakova and DellaPenna, 2003).

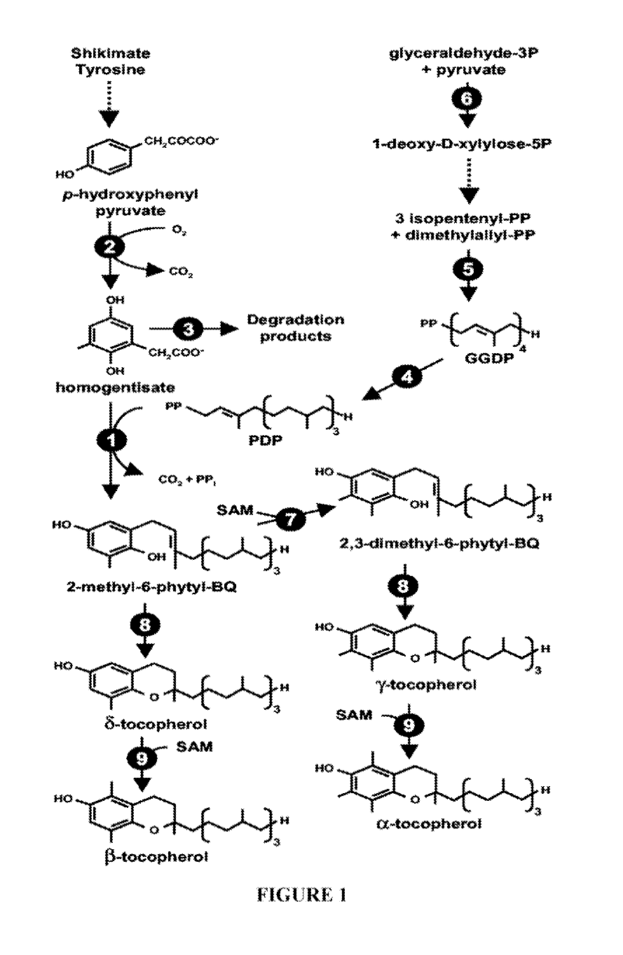

FIG. 2 shows the expression of an exemplary nucleic acid of the invention (OsVTE1) in rice. (A) Expression of OsVTE1 in rice seedlings in response to salt, hydrogen peroxide, drought, ABA, SA, ACC and cold stress. (B) Expression of OsVTE1 in root, stem, leaf, and spikelet determined by 28, 30, and 32 cycles of RT-PCR with gene-specific primers. (C)—(a) to (g)—OsVTE1 promoter-GUS expression in wild-type plants. (a) spikelet, (b) seed, (c) rachilla, (d) stamen, (e) pistil, (f) and (g) stem, (h) leaf.

FIG. 3 shows representative diagrams of particular vectors and the effect of expression of particular vectors in rice. (A) Schematic diagram of overexpression vector pBin438-OsVTE1. (B) Schematic diagram of RNA interference vector pZH01-VTE1-RNAi. (C) Schematic diagram of promoter GUS vector pBI121-OsVTE1-promoter. (D) Expression of OsVTE1 in plants transformed with pBin438-OsVTE1 (OX) plants determined by real-time PCR. (E) Expression of OsVTE1 in plants transformed with pZH01-VTE1-RNAi (RNAi) determined by real-time PCR.

FIG. 4 illustrates the salt stress tolerance of OsVTE1-RNAi and OsVTE1-OX transgenic plants. Plants were allowed to grow for three weeks under normal conditions, and then were subjected to treatment with 100 mM NaCl for ten days. After the stress was stopped, plants were permitted to recover for seven days. (A) OsVTE1-RNAi and OsVTE1-OX plants prior to treatment. (B) OsVTE1-RNAi and OsVTE1-OX plants after being subjected to 100 mM NaCl for ten days. (C) OsVTE1-RNAi and OsVTE1-OX plants after seven-day recovery period. (D) Chlorophyll content of OsVTE1-RNAi and OsVTE1-OX plants after treatment with 100 mM NaCl for ten days. White bars indicate control (CK) plants. Black bars indicate treated (NaCl) plants. (E) Survival ratio of OsVTE1-RNAi and OsVTE1-OX plants after seven-day recovery period.

FIG. 5 illustrates the drought stress tolerance of OsVTE1-RNAi and OsVTE1-OX transgenic plants. Plants were allowed to grow for four weeks under normal conditions, and then were subjected to drought stress for eight days. After stress was stopped, plants were re-watered over a fourteen-day period. (A) OsVTE1-RNAi and OsVTE1-OX plants prior to treatment. (B) OsVTE1-RNAi and OsVTE1-OX plants after being subjected to drought stress for eight days. (C) OsVTE1-RNAi and OsVTE1-OX plants on Day 4 of re-watering. (D) OsVTE1-RNAi and OsVTE1-OX plants on Day 14 of re-watering. (E) Survival ratio of OsVTE1-RNAi and OsVTE1-OX plants on Day 14 of re-watering.

FIG. 6 illustrates the difference in hydrogen peroxide production in OsVTE1-RNAi and OsVTE1-OX plants after being subjected to 100 mM NaCl for seven days. Hydrogen peroxide was detected using DAB (3,3-diaminobenzidine).

### DETAILED DESCRIPTION OF THE INVENTION

### Nucleic Acids and Proteins

As used herein, the terms “nucleic acid”, “polynucleotide”, “polynucleotide molecule”, “polynucleotide sequence” and plural variants are used interchangeably to refer to a wide variety of molecules, including single strand and double strand DNA and RNA molecules, cDNA sequences, genomic DNA sequences of exons and introns, chemically synthesized DNA and RNA sequences, and sense strands and corresponding antisense strands. Polynucleotides of the invention may also comprise known analogs of natural nucleotides that have similar properties as the reference natural nucleic acid.

As used herein, the terms “polypeptide”, “protein” and plural variants are used interchangeably and refer to a compound made up of a single chain of amino acids joined by peptide bonds. Polypeptides of the invention may comprise naturally occurring amino acids, synthetic amino acids, genetically encoded amino acids, non-genetically encoded amino acids, and combinations thereof. Polypeptides may include both L-form and D-form amino acids.

Representative non-genetically encoded amino acids include but are not limited to 2-aminoadipic acid; 3-aminoadipic acid; β-aminopropionic acid; 2-aminobutyric acid; 4-aminobutyric acid (piperidinic acid); 6-aminocaproic acid; 2-aminoheptanoic acid; 2-aminoisobutyric acid; 3-aminoisobutyric acid; 2-aminopimelic acid; 2,4-diaminobutyric acid; desmosine; 2,2′-diaminopimelic acid; 2,3-diaminopropionic acid; N-ethylglycine; N-ethylasparagine; hydroxylysine; allo-hydroxylysine; 3-hydroxyproline; 4-hydroxyproline; isodesmosine; allo-isoleucine; N-methylglycine (sarcosine); N-methylisoleucine; N-methylvaline; norvaline; norleucine; and ornithine.

Representative derivatized amino acids include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine.

Exemplary TC polynucleotides of the invention are set forth as SEQ ID NO: 1 and substantially identical sequences encoding proteins capable of altering a trait of a plant, for example, salt tolerance, drought tolerance, decreased chlorophyll loss in response to salt stress, decreased ROS production, and increased γ-tocopherol production. Another exemplary TC polynucleotide of the invention is set forth as SEQ ID NO: 3.

Exemplary TC polypeptides of the invention are set forth as SEQ ID NO: 2 and substantially identical proteins capable of altering a trait of a plant, for example, salt tolerance, drought tolerance, decreased chlorophyll loss in response to salt stress, decreased ROS production, and increased γ-tocopherol production.

Substantially identical sequences are those that have at least 70%, preferably at least 80%, preferably at least 85%, more preferably at least 90%, even more preferably at least 95%, and most preferably at least 99% nucleotide or amino acid residue identity, when compared and aligned for maximum correspondence using a sequence comparison algorithm or by visual inspection. Preferably, the substantial identity exists over a region of the sequences that is at least about 50 residues in length, more preferably over a region of at least about 100 residues, and most preferably the sequences are substantially identical over at least about 150 residues. In an especially preferred embodiment, the sequences are substantially identical over the entire length of the coding regions. Furthermore, substantially identical nucleic acids or proteins perform substantially the same function. Substantially identical sequences may be polymorphic sequences, i.e., alternative sequences or alleles in a population. An allelic difference may be as small as one base pair. Substantially identical sequences may also comprise mutagenized sequences, including sequences comprising silent mutations. A mutation may comprise one or more residue changes, a deletion of one or more residues, or an insertion of one or more additional residues. Substantially identical nucleic acids are also identified as nucleic acids that hybridize specifically to or hybridize substantially to a reference sequence (e.g., SEQ ID NO: 1).

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math., 2:482 (1981), by the homology alignment algorithm of Needleman & Wunsch, J. Mol. Biol., 48:443 (1970), by the search for similarity method of Pearson & Lipman, Proc. Natl. Acad. Sci. USA, 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see Ausubel et al., infra).

One example of an algorithm that is suitable for determining percent sequence identity and sequence similarity is the BLAST algorithm, which is described in Altschul et al., J. Mol. Biol., 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA, 89:10915 (1989)).

In addition to calculating percent sequence identity, the BLAST algorithm also performs a statistical analysis of the similarity between two sequences (see e.g., Karlin & Altschul, Proc. Natl. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

Another indication that two nucleic acid sequences are substantially identical is that the two molecules hybridize to each other under stringent conditions. Stringent conditions are those under which a nucleic acid probe will typically hybridize to its target sequence but to no other sequences when that sequence is present in a complex nucleic acid mixture (e.g., total cellular DNA or RNA). Stringent hybridization conditions and stringent hybridization wash conditions in the context of nucleic acid hybridization experiments such as Southern and Northern blot analyses are both sequence- and environment-dependent. An extensive guide to the hybridization of nucleic acids is found in Tijssen, Laboratory Techniques in Biochemistry and Molecular Biology-Hybridization with Nucleic Acid Probes, part I chapter 2, Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or Northern blot is 50% formamide with 1 mg of heparin at 42° C., with the hybridization being carried out overnight. An example of highly stringent wash conditions is 0.15 M NaCl at 72° C. for about 15 minutes. Another example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An exemplary medium stringency wash for a duplex of, e.g., more than 100 nucleotides, is 1×SSC at 45° C. for 15 minutes. An example low stringency wash for a duplex of, e.g., more than 100 nucleotides, is 4×-6×SSC at 40° C. for 15 minutes. For short probes (e.g., about 10 to 50 nucleotides), stringent conditions typically involve salt concentrations of less than about 1.0 M sodium ions, typically about 0.01 to 1.0 M sodium ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization.

The following are examples of hybridization and wash conditions that may be used to identify nucleotide sequences that are substantially identical to reference nucleotide sequences of the present invention. A substantially identical nucleotide sequence preferably hybridizes to a reference nucleotide sequence in 7% sodium dodecyl sulfate (SDS), 0.5 M NaPO₄, 1 mM EDTA at 50° C. with washing in 2×SSC, 0.1% SDS at 50° C., more preferably in 7% sodium dodecyl sulfate (SDS), 0.5 M NaPO₄, 1 mM EDTA at 50° C. with washing in 1×SSC, 0.1% SDS at 50° C., still more preferably in 7% sodium dodecyl sulfate (SDS), 0.5 M NaPO₄, 1 mM EDTA at 50° C. with washing in 0.5×SSC, 0.1% SDS at 50° C., even more preferably in 7% sodium dodecyl sulfate (SDS), 0.5 M NaPO₄, 1 mM EDTA at 50° C. with washing in 0.1×SSC, 0.1% SDS at 50° C., and most preferably in 7% sodium dodecyl sulfate (SDS), 0.5 M NaPO₄, 1 mM EDTA at 50° C. with washing in 0.1×SSC, 0.1% SDS at 65° C.

A further indication that two nucleic acid sequences or proteins are substantially identical is that the that proteins encoded by the nucleic acids are substantially identical, share an overall three-dimensional structure, are biologically functional equivalents, or are immunologically cross-reactive with, or specifically bind to, each other. Nucleic acid molecules that do not hybridize to each other under stringent conditions are still substantially identical if the corresponding proteins are substantially identical. This may occur, for example, when two nucleotide sequences comprise conservatively substituted variants as permitted by the genetic code. This also includes degenerate codon substitutions wherein the third position of one or more selected (or all) codons is substituted with mixed-base and/or deoxyinosine residues (see Batzer et al., Nucleic Acids Res., 19:5081 (1991); Ohtsuka et al., J. Biol. Chem., 260:2605-2608 (1985); and Rossolini et al. Mol. Cell Probes, 8:91-98 (1994)). However, both the polynucleotides and the polypeptides of the present invention may be conservatively substituted at one or more residues. Examples of conservative amino acid substitutions include the substitution of one non-polar (hydrophobic) residue such as isoleucine, valine, leucine or methionine for another; the substitution of one polar (hydrophilic) residue for another such as between arginine and lysine, between glutamine and asparagine, between glycine and serine; the substitution of one basic residue such as lysine, arginine or histidine for another; or the substitution of one acidic residue, such as aspartic acid or glutamic acid for another.

Nucleic acids of the invention also comprise nucleic acids complementary to SEQ ID NOs: 1 and 3, and subsequences and elongated sequences of SEQ ID NOs: 1 and 3 and complementary sequences thereof. Complementary sequences are two nucleotide sequences that comprise antiparallel nucleotide sequences capable of pairing with one another upon formation of hydrogen bonds between base pairs. Like other polynucleotides in accordance with the present invention, complementary sequences maybe substantially similar to one another as described previously. A particular example of a complementary nucleic acid segment is an antisense oligonucleotide.

A subsequence is a sequence of nucleic acids that comprises a part of a longer nucleic acid sequence. An exemplary subsequence is a probe or a primer. An elongated sequence is one in which nucleotides (or other analogous molecules) are added to a nucleic acid sequence. For example, a polymerase (e.g., a DNA polymerase) may add sequences at the 3′ terminus of the nucleic acid molecule. In addition, the nucleotide sequence may be combined with other DNA sequences, such as promoters, promoter regions, enhancers, polyadenylation signals, introns, additional restriction enzyme sites, multiple cloning sites, and other coding segments. Thus, the present invention also provides vectors comprising the disclosed nucleic acids, including vectors for recombinant expression, wherein a nucleic acid of the invention is operatively linked to a functional promoter. When operatively linked to a nucleic acid, a promoter is in functional combination with the nucleic acid such that the transcription of the nucleic acid is controlled and regulated by the promoter region. Vectors refer to nucleic acids capable of replication in a host cell, such as plasmids, cosmids, and viral vectors.

Polynucleotides of the present invention may be cloned, synthesized, altered, mutagenized, or combinations thereof. Standard recombinant DNA and molecular cloning techniques used to isolate nucleic acids are known in the art. Site-specific mutagenesis to create base pair changes, deletions, or small insertions is also known in the art (see e.g., Sambrook et al. (eds.) Molecular Cloning: A Laboratory Manual, 1989, Cold Spring Harbor Laboratory Press, Cold Spring Harbor, N.Y.; Silhavy et al., Experiments with Gene Fusions, 1984, Cold Spring Harbor Laboratory Press, Cold Spring Harbor, N.Y.; Glover & Hames, DNA Cloning: A Practical Approach, 2nd ed., 1995, IRL Press at Oxford University Press, Oxford/New York; Ausubel (ed.) Short Protocols in Molecular Biology, 3rd ed., 1995, Wiley, New York).

Isolated polypeptides of the invention may be purified and characterized using a variety of standard techniques that are known to the skilled artisan (see e.g., Schröder et al., The Peptides, 1965, Academic Press, New York; Bodanszky, Principles of Peptide Synthesis, 2nd rev. ed. 1993, Springer-Verlag, Berlin/N.Y.; Ausubel (ed.), Short Protocols in Molecular Biology, 3rd ed., 1995, Wiley, New York).

The present invention also encompasses methods for detecting a nucleic acid molecule that encodes a TC protein. Such methods may be used to detect gene variants or altered gene expression. Sequences detected by methods of the invention may detected, subcloned, sequenced, and further evaluated by any measure well known in the art using any method usually applied to the detection of a specific DNA sequence. Thus, the nucleic acids of the present invention may be used to clone genes and genomic DNA comprising the disclosed sequences. Alternatively, the nucleic acids of the present invention may be used to clone genes and genomic DNA of related sequences. Levels of a TC nucleic acid molecule may be measured, for example, using an RT-PCR assay (see e.g., Chiang, J. Chromatogr. A., 806:209-218 (1998) and references cited therein).

The present invention also encompasses genetic assays using TC nucleic acids for quantitative trait loci (QTL) analysis and to screen for genetic variants, for example by allele-specific oligonucleotide (ASO) probe analysis (Conner et al., Proc. Natl. Acad. Sci. USA, 80(1):278-282 (1983)), oligonucleotide ligation assays (OLAs) (Nickerson et al., Proc. Natl. Acad. Sci. USA, 87(22):8923-8927 (1990)), single-strand conformation polymorphism (SSCP) analysis (Orita et al., Proc. Natl. Acad. Sci. USA, 86(8):2766-2770 (1989)), SSCP/heteroduplex analysis, enzyme mismatch cleavage, direct sequence analysis of amplified exons (Kestila et al., Mol. Cell, 1(4):575-582 (1998); Yuan et al., Hum. Mutat., 14(5):440-446 (1999)), allele-specific hybridization (Stoneking et al., Am. J. Hum. Genet., 48(2):370-382 (1991)), and restriction analysis of amplified genomic DNA containing the specific mutation. Automated methods may also be applied to large-scale characterization of single nucleotide polymorphisms (Wang et al., Am. J. Physiol., 1998, 274(4 Pt 2):H1132-1140 (1992); Brookes, Gene, 234(2):177-186 (1999)). Preferred detection methods are non-electrophoretic, including, for example, the TAQMAN™ allelic discrimination assay, PCR-OLA, molecular beacons, padlock probes, and well fluorescence (see Landegren et al., Genome Res., 8:769-776 (1998) and references cited therein).

The present invention also encompasses functional fragments of a TC polypeptide, for example, fragments that have the ability to alter a plant trait similar to that of SEQ ID NO: 2. Functional polypeptide sequences that are longer than the disclosed sequences are also encompassed. For example, one or more amino acids may be added to the N-terminus or C-terminus of an antibody polypeptide. Such additional amino acids may be employed in a variety of applications, including but not limited to purification applications. Methods of preparing elongated proteins are known in the art.

The present invention also encompasses methods for detecting a polypeptide. Such methods can be used, for example, to determine levels of protein expression and correlate the level of expression with the presence or change in phenotype, trait, or level of expression in a different gene or gene product. In certain embodiments, the method involves an immunochemical reaction with an antibody that specifically recognizes a protein. Techniques for detecting such antibody-antigen conjugates or complexes are known in the art and include but are not limited to centrifugation, affinity chromatography and other immunochemical methods (see e.g., Ishikawa, Ultrasensitive and Rapid Enzyme Immunoassay, 1999, Elsevier, Amsterdam/New York, United States of America; Law, Immunoassay: A Practical Guide, 1996, Taylor & Francis, London/Bristol, Pa., United States of America; Liddell et al., Antibody Technology, 1995, Bios Scientific Publishers, Oxford, United Kingdom; and references cited therein).

### TC Expression Systems

An expression system refers to a host cell comprising a heterologous nucleic acid and the protein encoded by the heterologous nucleic acid. For example, a heterologous expression system may comprise a host cell transfected with a construct comprising a TC nucleic acid encoding a protein operatively linked to a promoter, or a cell line produced by introduction of TC nucleic acids into a host cell genome. The expression system may further comprise one or more additional heterologous nucleic acids relevant to TC function, such as targets of TC transcriptional activation or repression activity. These additional nucleic acids may be expressed as a single construct or multiple constructs.

A construct for expressing a TC protein may include a vector sequence and a TC nucleotide sequence, wherein the TC nucleotide sequence is operatively linked to a promoter sequence. A construct for recombinant TC expression may also comprise transcription termination signals and sequences required for proper translation of the nucleotide sequence. Preparation of an expression construct, including addition of translation and termination signal sequences, is known to one skilled in the art.

The promoter may be any polynucleotide sequence which shows transcriptional activity in the chosen plant cells, plant parts, or plants. The promoter may be native or analogous, or foreign or heterologous, to the plant host and/or to the DNA sequence of the invention. Where the promoter is native or endogenous to the plant host, it is intended that the promoter is found in the native plant into which the promoter is introduced. Where the promoter is foreign or heterologous to the DNA sequence of the invention, the promoter is not the native or naturally occurring promoter for the operably linked DNA sequence of the invention. The promoter may be inducible or constitutive. It may be naturally-occurring, may be composed of portions of various naturally-occurring promoters, or may be partially or totally synthetic. Guidance for the design of promoters is provided by studies of promoter structure, such as that of Harley et al., Nucleic Acids Res., 15:2343-61 (1987). Also, the location of the promoter relative to the transcription start may be optimized (see e.g., Roberts et al., Proc. Natl. Acad. Sci. USA, 76:760-4 (1979)). Many suitable promoters for use in plants are well known in the art.

For example, suitable constitutive promoters for use in plants include the promoters from plant viruses, such as the peanut chlorotic streak caulimovirus (PC1SV) promoter (U.S. Pat. No. 5,850,019); the 35S and 19S promoters from cauliflower mosaic virus (CaMV) (Odell et al., Nature, 313:810-812 (1985) and U.S. Pat. No. 5,352,605); the promoters of Chlorella virus methyltransferase genes (U.S. Pat. No. 5,563,328) and the full-length transcript promoter from figwort mosaic virus (FMV) (U.S. Pat. No. 5,378,619); the promoters from such genes as rice actin (McElroy et al., Plant Cell, 2:163-171 (1990)); ubiquitin (Binet et al., Plant Science, 79:87-94 (1991)), maize (Christensen et al., Plant Molec. Biol., 12:619-632 (1989)), and arabidopsis (Norris et al., Plant Molec. Biol., 21:895-906 (1993); and Christensen et al., Plant Mol. Biol., 18:675-689 (1982)); pEMU (Last et al., Theor. Appl. Genet., 81:581-588 (1991)); MAS (Velten et al., EMBO J., 3:2723-2730 (1984)); maize H3 histone (Lepetit et al., Mol. Gen. Genet., 1992, 231:276-285 (1992); and Atanassova et al., Plant J., 2(3):291-300 (1992)); Brassica napus ALS3 (PCT International Publication No. WO 97/41228); and promoters of various Agrobacterium genes (e.g., U.S. Pat. Nos. 4,771,002; 5,102,796; 5,182,200; and 5,428,147).

Suitable inducible promoters for use in plants include the promoter from the ACE1 system which responds to copper (Mett et al., Proc. Natl. Acad. Sci. USA, 90:4567-4571 (1993)); the promoter of the maize 1n2 gene which responds to benzenesulfonamide herbicide safeners (Hershey et al., Mol. Gen. Genetics, 227:229-237 (1991); and Gatz et al., Mol. Gen. Genetics, 243:32-38 (1994)); and the promoter of the Tet repressor from Tn10 (Gatz et al., Mol. Gen. Genet., 227:229-237 (1991)). Another inducible promoter for use in plants is one that responds to an inducing agent to which plants do not normally respond. An exemplary inducible promoter of this type is the inducible promoter from a steroid hormone gene, the transcriptional activity of which is induced by a glucocorticosteroid hormone (Schena et al., Proc. Natl. Acad. Sci. USA, 88:10421 (1991)) or the recent application of a chimeric transcription activator, XVE, for use in an estrogen receptor-based inducible plant expression system activated by estradiol (Zuo et al., Plant J., 24:265-273 (2000)). Other inducible promoters for use in plants are described in EP 332104, PCT International Publication Nos. WO 93/21334 and WO 97/06269. Promoters composed of portions of other promoters and partially or totally synthetic promoters can also be used (see e.g., Ni et al., Plant J., 7:661-676 (1995) and PCT International Publication No. WO 95/14098 describing such promoters for use in plants).

Tissue-specific or tissue-preferential promoters useful for the expression of the novel TC genes of the invention in plants, including the cotton rubisco promoter disclosed in U.S. Pat. No. 6,040,504; the rice sucrose synthase promoter disclosed in U.S. Pat. No. 5,604,121; and the cestrum yellow leaf curling virus promoter disclosed in PCT International Publication No. WO 01/73087. Chemically inducible promoters useful for directing the expression of TC genes in plants are disclosed in U.S. Pat. No. 5,614,395.

The promoter may include, or be modified to include, one or more enhancer elements to thereby provide for higher levels of transcription. Suitable enhancer elements for use in plants include the PC1SV enhancer element (U.S. Pat. No. 5,850,019), the CaMV 35S enhancer element (U.S. Pat. Nos. 5,106,739 and 5,164,316) and the FMV enhancer element (Maiti et al., Transgenic Res., 6:143-156 (1997)). See also PCT International Publication No. WO 96/23898.

Such constructs can contain a ‘signal sequence’ or ‘leader sequence’ to facilitate co-translational or post-translational transport of the peptide of interest to certain intracellular structures such as the chloroplast (or other plastid), endoplasmic reticulum, or Golgi apparatus, or to be secreted. For example, the construct can be engineered to contain a signal peptide to facilitate transfer of the peptide to the endoplasmic reticulum. A signal sequence is known or suspected to result in cotranslational or post-translational peptide transport across the cell membrane. In eukaryotes, this typically involves secretion into the Golgi apparatus, with some resulting glycosylation. A leader sequence refers to any sequence that, when translated, results in an amino acid sequence sufficient to trigger co-translational transport of the peptide chain to a sub-cellular organelle. Thus, this includes leader sequences targeting transport and/or glycosylation by passage into the endoplasmic reticulum, passage to vacuoles, plastids including chloroplasts, mitochondria, and the like. Plant expression cassettes may also contain an intron, such that mRNA processing of the intron is required for expression.

Such constructs can also contain 5′ and 3′ untranslated regions. A 3′ untranslated region is a polynucleotide located downstream of a coding sequence. Polyadenylation signal sequences and other sequences encoding regulatory signals capable of affecting the addition of polyadenylic acid tracts to the 3′ end of the mRNA precursor are 3′ untranslated regions. A 5′ untranslated region is a polynucleotide located upstream of a coding sequence.

The termination region may be native with the transcriptional initiation region, may be native with the sequence of the present invention, or may be derived from another source. Convenient termination regions are available from the Ti-plasmid of A. tumefaciens, such as the octopine synthase and nopaline synthase termination regions (see e.g., Guerineau et al., Mol. Gen. Genet., 262:141-144 (1991); Proudfoot, Cell, 64:671-674 (1991); Sanfacon et al., Genes Dev., 5:141-149 (1991); Mogen et al., Plant Cell, 2:1261-1272 (1990); Munroe et al., Gene, 91:151-158 (1990); Ballas et al., Nucleic Acids Res., 17:7891-7903 (1989); and Joshi et al., Nucleic Acid Res., 15:9627-9639 (1987)).

Where appropriate, the vector and TC sequences may be optimized for increased expression in the transformed host cell. That is, the sequences can be synthesized using host cell-preferred codons for improving expression, or may be synthesized using codons at a host-preferred codon usage frequency. Generally, the GC content of the polynucleotide will be increased (see e.g., Campbell et al., Plant Physiol., 92:1-11 (1990) for a discussion of host-preferred codon usage). Methods are known in the art for synthesizing host-preferred polynucleotides (see e.g., U.S. Pat. Nos. 6,320,100; 6,075,185; 5,380,831; and 5,436,391, U.S. Application Publication Nos. 20040005600 and 20010003849, and Murray et al., Nucleic Acids Res., 17:477-498 (1989).

In certain embodiments, polynucleotides of interest are targeted to the chloroplast for expression. In this manner, where the polynucleotide of interest is not directly inserted into the chloroplast, the expression cassette may additionally contain a polynucleotide encoding a transit peptide to direct the nucleotide of interest to the chloroplasts. Such transit peptides are known in the art (see e.g., Von Heijne et al., Plant Mol. Biol. Rep., 9:104-126 (1991); Clark et al., J. Biol. Chem., 264:17544-17550 (1989); Della-Cioppa et al., Plant Physiol., 84:965-968 (1987); Romer et al., Biochem. Biophys. Res. Commun., 196:1414-1421 (1993); and Shah et al., Science, 233:478-481 (1986)). The polynucleotides of interest to be targeted to the chloroplast may be optimized for expression in the chloroplast to account for differences in codon usage between the plant nucleus and this organelle. In this manner, the polynucleotides of interest may be synthesized using chloroplast-preferred codons (see e.g., U.S. Pat. No. 5,380,831).

A plant expression cassette (i.e., a TC open reading frame operatively linked to a promoter) can be inserted into a plant transformation vector, which allows for the transformation of DNA into a cell. Such a molecule may consist of one or more expression cassettes, and may be organized into more than one vector DNA molecule. For example, binary vectors are plant transformation vectors that utilize two non-contiguous DNA vectors to encode all requisite cis- and trans-acting functions for transformation of plant cells (Hellens et al., Trends in Plant Science, 5:446-451 (2000)).

A plant transformation vector comprises one or more DNA vectors for achieving plant transformation. For example, it is a common practice in the art to utilize plant transformation vectors that comprise more than one contiguous DNA segment. These vectors are often referred to in the art as binary vectors. Binary vectors as well as vectors with helper plasmids are most often used for Agrobacterium-mediated transformation, where the size and complexity of DNA segments needed to achieve efficient transformation is quite large, and it is advantageous to separate functions onto separate DNA molecules. Binary vectors typically contain a plasmid vector that contains the cis-acting sequences required for T-DNA transfer (such as left border and right border), a selectable marker that is engineered to be capable of expression in a plant cell, and a polynucleotide of interest (i.e., a polynucleotide engineered to be capable of expression in a plant cell for which generation of transgenic plants is desired).

For certain target species, different antibiotic or herbicide selectable markers may be preferred. Selection markers used routinely in transformation include the nptII gene, which confers resistance to kanamycin and related antibiotics (Messing & Vierra, Gene, 19:259-268 (1982); and Bevan et al., Nature, 304:184-187 (1983)), the bar gene, which confers resistance to the herbicide phosphinothricin (White et al., Nucl. Acids Res., 18:1062 (1990), and Spencer et al., Theor. Appl. Genet., 79:625-631 (1990)), the hph gene, which confers resistance to the antibiotic hygromycin (Blochinger & Diggelmann, Mol. Cell. Biol., 4:2929-2931 (1984)), the dhfr gene, which confers resistance to methotrexate (Bourouis et al., EMBO J., 2(7):1099-1104 (1983)), the EPSPS gene, which confers resistance to glyphosate (U.S. Pat. Nos. 4,940,935 and 5,188,642), and the mannose-6-phosphate isomerase gene, which provides the ability to metabolize mannose (U.S. Pat. Nos. 5,767,378 and 5,994,629).

Also present on this plasmid vector are sequences required for bacterial replication. The cis-acting sequences are arranged in a fashion to allow efficient transfer into plant cells and expression therein. For example, the selectable marker sequence and the sequence of interest are located between the left and right borders. Often a second plasmid vector contains the trans-acting factors that mediate T-DNA transfer from Agrobacterium to plant cells. This plasmid often contains the virulence functions (Vir genes) that allow infection of plant cells by Agrobacterium, and transfer of DNA by cleavage at border sequences and vir-mediated DNA transfer, as in understood in the art (Hellens et al., 2000). Several types of Agrobacterium strains (e.g., LBA4404, GV3101, EHA101, EHA105, etc.) can be used for plant transformation. The second plasmid vector is not necessary for introduction of polynucleotides into plants by other methods such as, e.g., microprojection, microinjection, electroporation, and polyethylene glycol.

In another embodiment, a nucleotide sequence of the present invention is directly transformed into a plastid genome. A major advantage of plastid transformation is that plastids are generally capable of expressing bacterial genes without substantial modification, and plastids are capable of expressing multiple open reading frames under control of a single promoter.

Plastid transformation technology is extensively described in U.S. Pat. Nos. 5,451,513, 5,545,817 and 5,545,818, in PCT International Application Publication WO 95/16783, and in McBride et al., Proc. Natl. Acad. Sci. USA, 91:7301-7305 (1994). The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rpsl2 genes conferring resistance to spectinomycin and/or streptomycin are utilized as selectable markers for transformation (Svab et al., Proc. Natl. Acad. Sci. USA, 87:8526-8530 (1990); Staub et al., Plant Cell, 4:39-45 (1992)). This results in stable homoplasmic transformants at a frequency of approximately one per 100 bombardments of target leaves. The presence of cloning sites between these markers allows creation of a plastid targeting vector for introduction of foreign genes (Staub et al., EMBO 1, 12:601-606 (1993)). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab et al., Proc. Natl. Acad. Sci. USA, 90:913-917 (1993)). Previously, this marker had been used successfully for high-frequency transformation of the plastid genome of the green alga Chlamydomonas reinhardtii (Goldschmidt-Clermont, Nucl. Acids Res., 19:4083-4089 (1991)). Other selectable markers useful for plastid transformation are known in the art. Typically, approximately 15-20 cell division cycles following transformation are required to reach a homoplastidic state. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% of the total soluble plant protein. In a preferred embodiment, a nucleotide sequence of the present invention is inserted into a plastid-targeting vector and transformed into the plastid genome of a desired plant host. Plants homoplastic for plastid genomes containing a nucleotide sequence of the present invention are obtained, and are preferentially capable of high expression of the nucleotide sequence.

### Host Cells

Host cells are cells into which a heterologous nucleic acid molecule of the invention may be introduced. Representative eukaryotic host cells include yeast and plant cells, as well as prokaryotic hosts such as E. coli and Bacillus subtilis. Preferred host cells for functional assays substantially or completely lack endogenous expression of a TC protein.

A host cell strain may be chosen which modulates the expression of the recombinant sequence, or modifies and processes the gene product in a specific manner. For example, different host cells have characteristic and specific mechanisms for the translational and post-translational processing and modification (e.g., glycosylation, phosphorylation of proteins). Appropriate cell lines or host cells may be chosen to ensure the desired modification and processing of the foreign protein expressed. For example, expression in a bacterial system may be used to produce a non-glycosylated core protein product, and expression in yeast will produce a glycosylated product.

The present invention further encompasses recombinant expression of a TC protein in a stable cell line. Methods for generating a stable cell line following transformation of a heterologous construct into a host cell are known in the art (see e.g., Joyner, Gene Targeting: A Practical Approach, 1993, Oxford University Press, Oxford/New York). Thus, transformed cells, tissues, and plants are understood to encompass not only the end product of a transformation process, but also transgenic progeny or propagated forms thereof.

### TC Knockout Plants

The present invention also provides TC knockout plants comprising a disruption of a TC locus. A disrupted gene may result in expression of an altered level of full-length TC protein or expression of a mutated variant TC protein. Plants with complete or partial functional inactivation of the TC gene may be generated, e.g., by expressing a mutant TC allele in the plant.

A knockout plant in accordance with the present invention may also be prepared using anti-sense, double-stranded RNA, or ribozyme TC constructs, driven by a universal or tissue-specific promoter to reduce levels of TC gene expression in somatic cells, thus achieving a “knock-down” phenotype. The present invention also provides the generation of plants with conditional or inducible inactivation of TC.

The present invention also encompasses transgenic plants with specific “knocked-in” modifications in the disclosed TC gene, for example to create an over-expression mutant having a dominant negative phenotype. Thus, “knocked-in” modifications include the expression of mutant alleles of the TC gene.

TC knockout plants may be prepared in monocot or dicot plants, such as maize, wheat, barley, rye, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugar beet, sunflower, rapeseed, clover, tobacco, carrot, cotton, alfalfa, rice, potato, eggplant, cucumber, Arabidopsis, and woody plants such as coniferous and deciduous trees. Rice, wheat, barley, oat, soybean and rye are particularly contemplated. As used herein, a plant refers to a whole plant, a plant organ (e.g., root, stem, leaf, flower bud, or embryo), a seed, a plant cell, a propagule, an embryo, other plant parts (e.g., protoplasts, pollen, pollen tubes, ovules, embryo sacs, zygotes) and progeny of the same. Plant cells can be differentiated or undifferentiated (e.g., callus, suspension culture cells, protoplasts, leaf cells, root cells, phloem cells, pollen).

For preparation of a TC knockout plant, introduction of a polynucleotide into plant cells is accomplished by one of several techniques known in the art, including but not limited to electroporation or chemical transformation (see e.g., Ausubel, ed. (1994) Current Protocols in Molecular Biology, John Wiley and Sons, Inc., Indianapolis, Ind.). Markers conferring resistance to toxic substances are useful in identifying transformed cells (having taken up and expressed the test polynucleotide sequence) from non-transformed cells (those not containing or not expressing the test polynucleotide sequence). In one aspect of the invention, genes are useful as a marker to assess introduction of DNA into plant cells. Transgenic plants, transformed plants, or stably transformed plants, or cells, tissues or seed of any of the foregoing, refer to plants that have incorporated or integrated exogenous polynucleotides into the plant cell. Stable transformation refers to introduction of a polynucleotide construct into a plant such that it integrates into the genome of the plant and is capable of being inherited by progeny thereof.

In general, plant transformation methods involve transferring heterologous DNA into target plant cells (e.g., immature or mature embryos, suspension cultures, undifferentiated callus, protoplasts, etc.), followed by applying a maximum threshold level of appropriate selection (depending on the selectable marker gene) to recover the transformed plant cells from a group of untransformed cell mass. Explants are typically transferred to a fresh supply of the same medium and cultured routinely. Subsequently, the transformed cells are differentiated into shoots after placing on regeneration medium supplemented with a maximum threshold level of selecting agent (i.e., temperature and/or herbicide). The shoots are then transferred to a selective rooting medium for recovering rooted shoot or plantlet. The transgenic plantlet then grow into mature plant and produce fertile seeds (see e.g., Hiei et al., Plant J., 6:271-282 (1994); and Ishida et al., Nat. Biotechnol., 14:745-750 (1996)). A general description of the techniques and methods for generating transgenic plants are found in Ayres et al., CRC Crit. Rev. Plant Sci., 13:219-239 (1994); and Bommineni et al., Maydica, 42:107-120 (1997). Since the transformed material contains many cells, both transformed and non-transformed cells are present in any piece of subjected target callus or tissue or group of cells. The ability to kill non-transformed cells and allow transformed cells to proliferate results in transformed plant cultures. Often, the ability to remove non-transformed cells is a limitation to rapid recovery of transformed plant cells and successful generation of transgenic plants. Subsequently, molecular and biochemical methods can be used for confirming the presence of the integrated nucleotide(s) of interest in the genome of transgenic plant.

Generation of transgenic plants may be performed by one of several methods, including but not limited to introduction of heterologous DNA by Agrobacterium into plant cells (Agrobacterium-mediated transformation), bombardment of plant cells with heterologous foreign DNA adhered to particles, and various other non-particle direct-mediated methods to transfer DNA (see e.g., Hiei et al., Plant J., 6:271-282 (1994); Ishida et al., Nat. Biotechnol., 14:745-750 (1996); Ayres et al., CRC Crit. Rev. Plant Sci., 13:219-239 (1994); and Bommineni et al., Maydica, 1997, 42:107-120 (1997)).

There are three common methods to transform plant cells with Agrobacterium. The first method is co-cultivation of Agrobacterium with cultured isolated protoplasts. This method requires an established culture system that allows culturing protoplasts and plant regeneration from cultured protoplasts. The second method is transformation of cells or tissues with Agrobacterium. This method requires (a) that the plant cells or tissues can be transformed by Agrobacterium and (b) that the transformed cells or tissues can be induced to regenerate into whole plants. The third method is transformation of seeds, apices or meristems with Agrobacterium. This method requires micropropagation.

The efficiency of transformation by Agrobacterium may be enhanced by using a number of methods known in the art. For example, the inclusion of a natural wound response molecule such as acetosyringone (AS) to the Agrobacterium culture has been shown to enhance transformation efficiency with Agrobacterium tumefaciens (Shahla et al., Plant Molec. Biol, 8:291-298 (1987)). Alternatively, transformation efficiency may be enhanced by wounding the target tissue to be transformed. Wounding of plant tissue may be achieved, for example, by punching, maceration, bombardment with microprojectiles (see e.g., Bidney et al., Plant Molec. Biol., 18:301-313 (1992).

In one embodiment, the plant cells are transfected with vectors via particle bombardment (i.e., with a gene gun). Particle mediated gene transfer methods are known in the art, are commercially available, and include, but are not limited to, the gas driven gene delivery instrument described in U.S. Pat. No. 5,584,807. This method involves coating the polynucleotide sequence of interest onto heavy metal particles, and accelerating the coated particles under the pressure of compressed gas for delivery to the target tissue.

Other particle bombardment methods are also available for the introduction of heterologous polynucleotide sequences into plant cells. Generally, these methods involve depositing the polynucleotide sequence of interest upon the surface of small, dense particles of a material such as gold, platinum, or tungsten. The coated particles are themselves then coated onto either a rigid surface, such as a metal plate, or onto a carrier sheet made of a fragile material such as mylar. The coated sheet is then accelerated toward the target biological tissue. The use of the flat sheet generates a uniform spread of accelerated particles that maximizes the number of cells receiving particles under uniform conditions, resulting in the introduction of the polynucleotide sample into the target tissue.

Specific initiation signals may also be used to achieve more efficient translation of sequences encoding the polypeptide of interest. Such signals include the ATG initiation codon and adjacent sequences. In cases where sequences encoding the polypeptide of interest, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers that are appropriate for the particular cell system that is used, such as those described in the literature (Scharf et al., Results Probl. Cell Differ., 20:125 (1994)).

The cells that have been transformed may be grown into plants in accordance with conventional ways (see e.g., McCormick et al., Plant Cell Rep., 5:81-84 (1986)). These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as transgenic seed) having a polynucleotide of the invention, for example, an expression cassette of the invention, stably incorporated into their genome.

Transgenic plants of the invention can be homozygous for the added polynucleotides; i.e., a transgenic plant that contains two added sequences, one sequence at the same locus on each chromosome of a chromosome pair. A homozygous transgenic plant can be obtained by sexually mating (selfing) an independent segregant transgenic plant that contains the added sequences according to the invention, germinating some of the seed produced and analyzing the resulting plants produced for enhanced enzyme activity (i.e., herbicide resistance) and/or increased plant yield relative to a control (native, non-transgenic) or an independent segregant transgenic plant.

It is to be understood that two different transgenic plants can also be mated to produce offspring that contain two independently segregating added, exogenous polynucleotides.

Selfing of appropriate progeny can produce plants that are homozygous for all added, exogenous polynucleotides that encode a polypeptide of the present invention. Back-crossing to a parental plant and outcrossing with a non-transgenic plant are also contemplated.

Following introduction of DNA into plant cells, the transformation or integration of the polynucleotide into the plant genome is confirmed by various methods such as analysis of polynucleotides, polypeptides and metabolites associated with the integrated sequence.

### TC Inhibitors

The present invention further discloses assays to identify TC binding partners and TC inhibitors. TC antagonists/inhibitors are agents that alter chemical and biological activities or properties of a TC protein. Methods of identifying inhibitors involve assaying a reduced level or quality of TC function in the presence of one or more agents. Exemplary TC inhibitors include small molecules as well as biological inhibitors as described herein below.

As used herein, the term “agent” refers to any substance that potentially interacts with a TC nucleic acid or protein, including any of synthetic, recombinant, or natural origin. An agent suspected to interact with a protein may be evaluated for such an interaction using the methods disclosed herein.

Exemplary agents include but are not limited to peptides, proteins, nucleic acids, small molecules (e.g., chemical compounds), antibodies or fragments thereof, nucleic acid-protein fusions, any other affinity agent, and combinations thereof. An agent to be tested may be a purified molecule, a homogenous sample, or a mixture of molecules or compounds.

A small molecule refers to a compound, for example an organic compound, with a molecular weight of less than about 1,000 daltons, more preferably less than about 750 daltons, still more preferably less than about 600 daltons, and still more preferably less than about 500 daltons. A small molecule also preferably has a computed log octanol-water partition coefficient in the range of about −4 to about +14, more preferably in the range of about −2 to about +7.5.

Exemplary nucleic acids that may be used to disrupt TC function include antisense RNA and small interfering RNAs (siRNAs) (see e.g., U.S. Application Publication No. 20060095987). These inhibitory molecules may be prepared based upon the TC gene sequence and known features of inhibitory nucleic acids (see e.g., Van der Krol et al., Plant Cell, 2:291-299 (1990); Napoli et al., Plant Cell, 2:279-289 (1990); English et al., Plant Cell, 8:179-188 (1996); and Waterhouse et al., Nature Rev. Genet., 2003, 4:29-38 (2003).

Agents may be obtained or prepared as a library or collection of molecules. A library may contain a few or a large number of different molecules, varying from about ten molecules to several billion molecules or more. A molecule may comprise a naturally occurring molecule, a recombinant molecule, or a synthetic molecule. A plurality of agents in a library may be assayed simultaneously. Optionally, agents derived from different libraries may be pooled for simultaneous evaluation.

Representative libraries include but are not limited to a peptide library (U.S. Pat. Nos. 6,156,511, 6,107,059, 5,922,545, and 5,223,409), an oligomer library (U.S. Pat. Nos. 5,650,489 and 5,858,670), an aptamer library (U.S. Pat. Nos. 7,338,762; 7,329,742; 6,949,379; 6,180,348; and 5,756,291), a small molecule library (U.S. Pat. Nos. 6,168,912 and 5,738,996), a library of antibodies or antibody fragments (U.S. Pat. Nos. 6,174,708, 6,057,098, 5,922,254, 5,840,479, 5,780,225, 5,702,892, and 5,667,988), a library of nucleic acid-protein fusions (U.S. Pat. No. 6,214,553), and a library of any other affinity agent that may potentially bind to a TC protein.

A library may comprise a random collection of molecules. Alternatively, a library may comprise a collection of molecules having a bias for a particular sequence, structure, or conformation, for example, as for inhibitory nucleic acids (see e.g., U.S. Pat. Nos. 5,264,563 and 5,824,483). Methods for preparing libraries containing diverse populations of various types of molecules are known in the art, for example as described in U.S. patents cited herein above. Numerous libraries are also commercially available.

A control level or quality of TC activity refers to a level or quality of wild type TC activity, for example, when using a recombinant expression system comprising expression of SEQ ID NOs: 1 and 3. When evaluating the inhibiting capacity of an agent, a control level or quality of TC activity comprises a level or quality of activity in the absence of the agent. A control level may also be established by a phenotype or other measurable trait.

Methods of identifying TC inhibitors also require that the inhibiting capacity of an agent be assayed. Assaying the inhibiting capacity of an agent may comprise determining a level of TC gene expression; determining DNA binding activity of a recombinantly expressed TC protein; determining an active conformation of a TC protein; or determining a change in a trait in response to binding of a TC inhibitor (e.g., increased salt tolerance, increased drought tolerance, decreased chlorophyll loss in response to salt stress, decreased ROS production, and increased γ-tocopherol production). In particular embodiments, a method of identifying a TC inhibitor may comprise (a) providing a cell, plant, or plant part expressing a TC protein; (b) contacting the cell, plant, or plant part with an agent; (c) examining the cell, plant, or plant part for a change in a trait as compared to a control; and (d) selecting an agent that induces a change in the trait as compared to a control. Any of the agents so identified in the disclosed inhibitory or binding assays (see hereinafter) may be subsequently applied to a cell, plant or plant part as desired to effectuate a change in that cell, plant or plant part. For example, disruption of a TC gene (e.g., SEQ ID NOs: 1 and 3) or inhibition of a TC polynucleotide or polypeptide (e.g., SEQ ID NO: 2) would alter one or more plant traits in a desirable way (e.g., increase drought tolerance).

The present invention also encompasses a rapid and high throughput screening method that relies on the methods described herein. This screening method comprises separately contacting a TC protein with a plurality of agents. In such a screening method the plurality of agents may comprise more than about 10⁴ samples, or more than about 10⁵ samples, or more than about 10⁶ samples.

The in vitro and cellular assays of the invention may comprise soluble assays, or may further comprise a solid phase substrate for immobilizing one or more components of the assay. For example, a TC protein, or a cell expressing a TC protein, may be bound directly to a solid state component via a covalent or non-covalent linkage. Optionally, the binding may include a linker molecule or tag that mediates indirect binding of a TC protein to a substrate.

### TC Binding Assays

The present invention also encompasses methods of identifying of a TC inhibitor by determining specific binding of a substance (e.g., an agent described previously) to a TC protein. For example, a method of identifying a TC binding partner may comprise: (a) providing a TC protein of SEQ ID NO: 2; (b) contacting the TC protein with one or more agents under conditions sufficient for binding; (c) assaying binding of the agent to the isolated TC protein; and (d) selecting an agent that demonstrates specific binding to the TC protein. Specific binding may also encompass a quality or state of mutual action such that binding of an agent to a TC protein is inhibitory.

Specific binding refers to a binding reaction which is determinative of the presence of the protein in a heterogeneous population of proteins and other biological materials. The binding of an agent to a TC protein may be considered specific if the binding affinity is about 1×10⁴M⁻¹ to about 1×10⁶M⁻¹ or greater. Specific binding also refers to saturable binding. To demonstrate saturable binding of an agent to a TC protein, Scatchard analysis may be carried out as described, for example, by Mak et al., J. Biol. Chem., 264:21613-21618 (1989).

Several techniques may be used to detect interactions between a TC protein and an agent without employing a known competitive inhibitor. Representative methods include, but are not limited to, Fluorescence Correlation Spectroscopy, Surface-Enhanced Laser Desorption/Ionization Time-Of-Flight Spectroscopy, and BIACORE® technology, each technique described herein below. These methods are amenable to automated, high-throughput screening.

Fluorescence Correlation Spectroscopy (FCS) measures the average diffusion rate of a fluorescent molecule within a small sample volume. The sample size may be as low as 10³ fluorescent molecules and the sample volume as low as the cytoplasm of a single bacterium. The diffusion rate is a function of the mass of the molecule and decreases as the mass increases. FCS may therefore be applied to protein-ligand interaction analysis by measuring the change in mass and therefore in diffusion rate of a molecule upon binding. In a typical experiment, the target to be analyzed (e.g., a TC protein) is expressed as a recombinant protein with a sequence tag, such as a poly-histidine sequence, inserted at the N-terminus or C-terminus. The expression is mediated in a host cell, such as E. coli, yeast, Xenopus oocytes, or mammalian cells. The protein is purified using chromatographic methods. For example, the poly-histidine tag may be used to bind the expressed protein to a metal chelate column such as Ni²⁺ chelated on iminodiacetic acid agarose. The protein is then labeled with a fluorescent tag such as carboxytetramethylrhodamine or BODIPY™ reagent (available from Molecular Probes of Eugene, Oreg.). The protein is then exposed in solution to the potential ligand, and its diffusion rate is determined by FCS using instrumentation available from Carl Zeiss, Inc. (Thornwood of New York, N.Y.). Ligand binding is determined by changes in the diffusion rate of the protein.

Surface-Enhanced Laser Desorption/Ionization (SELDI) was developed by Hutchens & Yip, Rapid Commun. Mass Spectrom., 1993, 7:576-580. When coupled to a time-of-flight mass spectrometer (TOF), SELDI provides a technique to rapidly analyze molecules retained on a chip. It may be applied to ligand-protein interaction analysis by covalently binding the target protein, or portion thereof, on the chip and analyzing by mass spectrometry the small molecules that bind to this protein (Worrall et al., Anal Chem., 1998, 70(4):750-756 (1998)). In a typical experiment, a target protein (e.g., a TC protein) is recombinantly expressed and purified. The target protein is bound to a SELDI chip either by utilizing a poly-histidine tag or by other interaction such as ion exchange or hydrophobic interaction. A chip thus prepared is then exposed to the potential ligand via, for example, a delivery system able to pipet the ligands in a sequential manner (autosampler). The chip is then washed in solutions of increasing stringency, for example a series of washes with buffer solutions containing an increasing ionic strength. After each wash, the bound material is analyzed by submitting the chip to SELDI-TOF. Ligands that specifically bind a target protein are identified by the stringency of the wash needed to elute them.

BIACORE® relies on changes in the refractive index at the surface layer upon binding of a ligand to a target protein (e.g., a TC protein) immobilized on the layer. In this system, a collection of small ligands is injected sequentially in a 2-5 microliter cell, wherein the target protein is immobilized within the cell. Binding is detected by surface plasmon resonance (SPR) by recording laser light refracting from the surface. In general, the refractive index change for a given change of mass concentration at the surface layer is practically the same for all proteins and peptides, allowing a single method to be applicable for any protein. In a typical experiment, a target protein is recombinantly expressed, purified, and bound to a BIACORE® chip. Binding may be facilitated by utilizing a poly-histidine tag or by other interaction such as ion exchange or hydrophobic interaction. A chip thus prepared is then exposed to one or more potential ligands via the delivery system incorporated in the instruments sold by Biacore (Uppsala, Sweden) to pipet the ligands in a sequential manner (autosampler). The SPR signal on the chip is recorded and changes in the refractive index indicate an interaction between the immobilized target and the ligand. Analysis of the signal kinetics of on rate and off rate allows the discrimination between non-specific and specific interaction (see also Homola et al., Sensors and Actuators, 54:3-15 (1999) and references therein).

### Conformational Assays

The present invention also encompasses methods of identifying TC binding partners and inhibitors that rely on a conformational change of a TC protein when bound by or otherwise interacting with a substance (e.g., an agent described previously). For example, application of circular dichroism to solutions of macromolecules reveals the conformational states of these macromolecules. The technique may distinguish random coil, alpha helix, and beta chain conformational states.

To identify inhibitors of a TC protein, circular dichroism analysis may be performed using a recombinantly expressed TC protein. A TC protein is purified, for example by ion exchange and size exclusion chromatography, and mixed with an agent. The mixture is subjected to circular dichroism. The conformation of a TC protein in the presence of an agent is compared to a conformation of a TC protein in the absence of the agent. A change in conformational state of a TC protein in the presence of an agent identifies a TC binding partner or inhibitor. Representative methods are described in U.S. Pat. Nos. 5,776,859 and 5,780,242. Antagonistic activity of the inhibitor may be assessed using functional assays, such assaying nitrate content, nitrate uptake, lateral root growth, or plant biomass, as described herein.

In accordance with the disclosed methods, cells expressing TC may be provided in the form of a kit useful for performing an assay of TC function. For example, a kit for detecting a TC may include cells transfected with DNA encoding a full-length TC protein and a medium for growing the cells.

Assays of TC activity that employ transiently transfected cells may include a marker that distinguishes transfected cells from non-transfected cells. A marker may be encoded by or otherwise associated with a construct for TC expression, such that cells are simultaneously transfected with a nucleic acid molecule encoding TC and the marker. Representative detectable molecules that are useful as markers include but are not limited to a heterologous nucleic acid, a protein encoded by a transfected construct (e.g., an enzyme or a fluorescent protein), a binding protein, and an antigen.

Assays employing cells expressing recombinant TC or plants expressing TC may additionally employ control cells or plants that are substantially devoid of native TC and, optionally, proteins substantially similar to a TC protein. When using transiently transfected cells, a control cell may comprise, for example, an untransfected host cell. When using a stable cell line expressing a TC protein, a control cell may comprise, for example, a parent cell line used to derive the TC-expressing cell line.

### Anti-TC Antibodies

In another aspect of the invention, a method is provided for producing an antibody that specifically binds a TC protein. According to the method, a full-length recombinant TC protein is formulated so that it may be used as an effective immunogen, and used to immunize an animal so as to generate an immune response in the animal. The immune response is characterized by the production of antibodies that may be collected from the blood serum of the animal.

An antibody is an immunoglobulin protein, or antibody fragments that comprise an antigen binding site (e.g., Fab, modified Fab, Fab′, F(ab′)₂ or Fv fragments, or a protein having at least one immunoglobulin light chain variable region or at least one immunoglobulin heavy chain region). Antibodies of the invention include diabodies, tetrameric antibodies, single chain antibodies, tetravalent antibodies, multispecific antibodies (e.g., bispecific antibodies), and domain-specific antibodies that recognize a particular epitope. Cell lines that produce anti-TC antibodies are also encompassed by the invention.

Specific binding of an antibody to a TC protein refers to preferential binding to a TC protein in a heterogeneous sample comprising multiple different antigens. Substantially lacking binding describes binding of an antibody to a control protein or sample, i.e., a level of binding characterized as non-specific or background binding. The binding of an antibody to an antigen is specific if the binding affinity is at least about 10⁻⁷M or higher, such as at least about 10⁻⁸M or higher, including at least about 10⁻⁹M or higher, at least about 10⁻¹¹M or higher, or at least about 10⁻¹²M or higher.

TC antibodies prepared as disclosed herein may be used in methods known in the art relating to the expression and activity of TC proteins, e.g., for cloning of nucleic acids encoding a TC protein, immunopurification of a TC protein, and detecting a TC protein in a plant sample, and measuring levels of a TC protein in plant samples. To perform such methods, an antibody of the present invention may further comprise a detectable label, including but not limited to a radioactive label, a fluorescent label, an epitope label, and a label that may be detected in vivo. Methods for selection of a label suitable for a particular detection technique, and methods for conjugating to or otherwise associating a detectable label with an antibody are known to one skilled in the art.

### EXAMPLES

The invention is now described with reference to the following Examples. These Examples are provided for the purpose of illustration only, and the invention is not limited to these Examples, but rather encompasses all variations which are evident as a result of the teachings provided herein.

### Example 1

### RNA Isolation, RT-PCR and Real-Time Quantitative RT-PCR Analysis

The expression pattern of OsVTE1 (Os02g0276500) was investigated in rice seedlings (Oryza sativa, subsp. japonica cv. Taipei309; also referred to herein as TP309) using reverse transcription RT-PCR under various treatments. Total RNA isolation was performed following the method description by Zhang et al. (1999b). First-strand cDNA synthesis was primed with Oligo(dT)₁₅ and catalyzed with M-MLV reverse transcriptase (Promega) at 37° C. for 1.5 hours. Reaction products were diluted 5-fold and used as templates for RT-PCR and real-time quantitative RT-PCR analysis.

Gene-specific primers, RT-OsVTE1 (5′-AGGGCCTATTCATCTCTACC-3′; SEQ ID NO: 4) and RT-OsVTE2 (5′-GGTGTCCATTCCCGAGTGCAGGCA-3′; SEQ ID NO: 5), were used for RT-PCR. Real-time quantitative RT-PCR analysis was performed using SYBR® Green PCR, an ABI PRISM® 7000 sequence detection system (Applied Biosystems), and gene-specific primers, Rtime1 (5′-TGCAATGTCTTCTCAGGCGC-3′; SEQ ID NO: 6) and Rtime2 (5′-GCTTCTATTTCAACCAGATG-3′; SEQ ID NO: 7). Quantitative RT-PCR results were analyzed with Microsoft Excel® software.

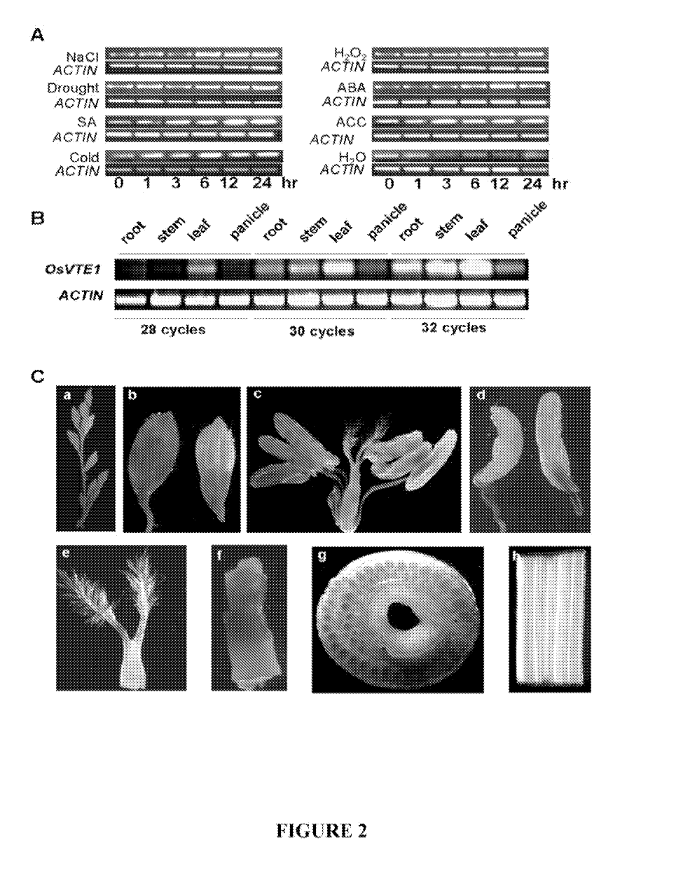

As shown in FIG. 2A, OsVTE1 was significantly induced by several abiotic stresses including 200 mM NaCl, drought, −4° C., 100 μM H₂O₂, 100 μM salicylic acid (SA), and 100 μM abscisic acid (ABA). OsVTE1 transcription levels also continually increased over a 24-hour period. However, 100 μM ACC and H₂O treatments had no effect on OsVTE1 transcription, providing evidence that OsVTE1 plays a role in plants' adaptation to abiotic stresses.

To further determine the tissue specificity of OsVTE1 expression, total RNAs were extracted from root, stem, leaf and spikelets of TP309 respectively. OsVTE1 was strongly expressed in leaf and stem, and also could be detected in the root and panicle by RT-PCR (FIG. 2B).

### Example 2

### GUS Expression Analysis

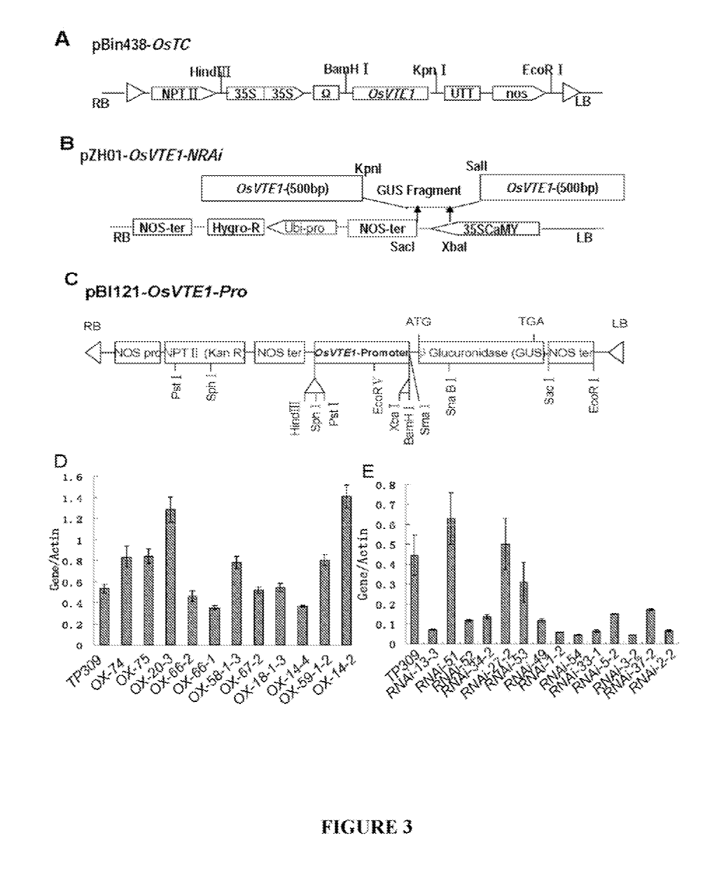

As shown in FIG. 3C, the OsVTE1 gene promoter (a 1.3 kB fragment upstream of the translation start site) was amplified using primers 5′-CCAAGCTTGCACGACCATAGG CGTGGGT-3′ (SEQ ID NO: 8) and 5′-GCTCTAGAGCTGATGCTGCGGGCGGGCA-3′ (SEQ ID NO: 9) and cloned into the HindI and BamHI sites of pBI121 containing a β-glucuronidase (GUS) reporter gene. The resulting construct was transferred into TP309 rice by Agrobacterium tumefaciens-mediated transformation as described by Hiei et al. (1994). GUS assays were performed at different developmental stages according to the method of Jefferson et al. (1987), and approximately fifteen independent T2 positive transgenic lines were used for the GUS staining assay shown in FIGS. 2C(a)-(h). Consistent with the results obtained from the RT-PCR assays, GUS was expressed in the stem, spikelet and leaf.

### Example 3

### Construction of Overexpression and RNAi Vectors

As shown in FIG. 3A, the overexpression vector pBin438-OsTC was created by amplifying the full-length OsVTE1 (Os02g0276500) cDNA sequence using RT-PCR and gene-specified primer pairs 5′-CGGGGTACCAGGGCCTATTCATCTCTACC-3′ (SEQ ID NO: 10) and 5′-CGCGGATCCAGCATCAGCATGGACCTCGC-3′ (SEQ ID NO: 11). The full length sequence was cloned into the Kpn I and BamH I sites of the binary vector pBIN438 as described previously (Xie et al., 2002). Gene expression was driven by two copies of the 35S promoter, and the tobacco mosaic virus omega sequence was included downstream of the 35S promoter to enhance translation efficiency. The construct was introduced into Agrobacterium tumefaciens strain AGL1 and then transformed into TP309 rice as described by Hiei et al. (1994). A vector for overexpressing SEQ ID NO: 3 in corn is created using a similar protocol.

As shown in FIG. 3B, the RNAi vector pZH01-OsVTE1 was constructed by amplifying a 489-bp fragment of OsVTE1 (from 409 by to 897 bp) using specific primers RNAiF (5′-TGCTCTAGAGAGCTCCAGTTCACCGAGAAATCC-3′; SEQ ID NO: 12) and RNAiR (5′-ACCGTCGACGAGCTCAGATGCGCC TGAGAAGAC-3′; SEQ ID NO: 13). The former primer contains XbaI and SacI sites, and the latter primer contains Sail and SacI sites. The sense and antisense fragments were assembled via respective restriction sites into vector pZH01. The RNAi vector pZH01-OsVTE1 contains the 35S promoter, the NOS terminator, and a fragment of GUS gene between the two inserted 489-bp fragments of the OsVTE1 gene.

### Example 4

### Effect of Salt Stress on TP309 Rice

TP309 rice were transformed using both pBin438-OsTC and pZH01-OsVTE1 vectors as described in Example 2. Transgenic OsVTE1-OX, OsVTE1-RNAi, and control TP309 plants were sown in pots (8×10 cm) containing vermiculite soaked with water. All plants were grown under white fluorescent light (600 mmol/m²/s, 12 h light period/day) at 28° C. and in 75% relative humidity. Three-week-old seedlings were transferred into a 100 mM NaCl solution for ten days, rinsed three times with water and then allowed to recover. A similar protocol is used to evaluate the effect of salt stress on transgenic corn transformed with an overexpression vector comprising SEQ ID NO: 3.

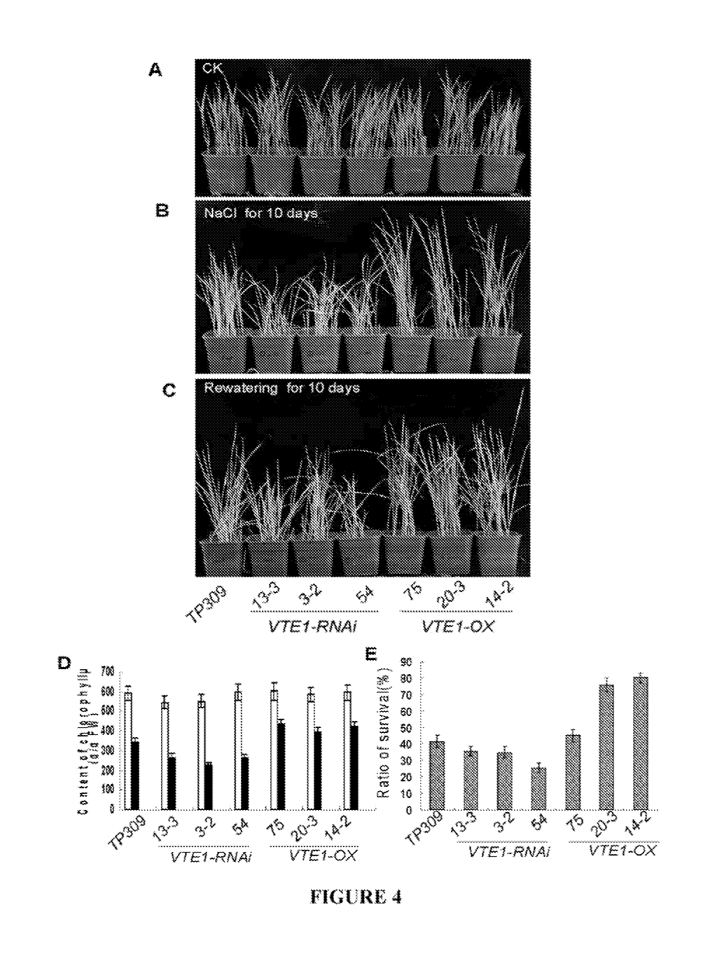

Transgenic plants were checked for expression of OsVTE1 by Real-Time PCR (FIGS. 3D and 3E respectively), and transgenic plants failed to exhibit any macroscopic differences from control plants when grown under normal greenhouse conditions (CK; FIG. 4A). The selfed progeny of three independent transgenic OsVTE1-OX (i.e., OsTC-OX-14-2, 20-3 and 75) and OsVTE1-RNAi (i.e., OsTC-RNAi-3-2, 13-3 and 64) lines were used for phenotype and physiological assays.

After ten days exposure to 100 mM NaCl, clear phenotypic differences were evident between the transgenic and control plants. Nearly 80% leaves of all three OsVTE1-RNAi lines were wilted. Control plants fared much better, but did still not grow nearly as well as OsVTE1-OX plants (FIG. 4B). Plants were subsequently rinsed three times with water, and then allowed to recover. Consistent with FIGS. 4C and 4E, OsVTE1-OX plants recovered more quickly than either control plants or OsVTE1-RNAi plants and had a significantly greater survival rate after seven days (FIG. 4E).

The effect of salt stress on chlorophyll content in TP309 rice was also evaluated. After treatment with 100 mM NaCl for ten days, approximately 0.1 g of leaves were excised from plants and immersed in an extract solution of ethanol:acetone:water (45:45:10) at room temperature until the leaves were bleached. The absorbance measurements from the extracts were read at 647 nm and 665 nm. The total chlorophyll content was calculated (Inskeep and Bloom, 1985) and expressed as mgl·g-1 FW. A similar protocol is used to evaluate the effect of salt stress on chlorophyll content in transgenic corn transformed with an overexpression vector comprising SEQ ID NO: 3.

As shown in FIG. 4D, salt stress decreased chlorophyll content in all plants, but the loss was significantly lessened in OsVTE1-OX plants. Compared to an approximately 40% loss in control plants and 50-60% loss in OsVTE1-RNAi plants, OsVTE1-OX plants lost only about 26% of chlorophyll.

Salt stress is also known to induce accumulation of reactive oxygen species (ROS) such as H₂O₂ (Hasegawa et al., 2000). After treatment with 100 mM NaCl for seven days, leaves of various plants were excised and immersed in a 1% solution of 3′,3′-diamino benzidine (DAB) in Tris-HCl buffer (pH 6.5). After vacuum-infiltration for 30 minutes, samples were incubated at room temperature for 20 hours in the dark. Leaves were subsequently bleached by immersion in boiling ethanol to more clearly visualize brown spots on the leaves, which are characteristic of the DAB/hydrogen peroxide reaction. A similar protocol is used to evaluate the effect of salt stress on reactive oxygen species generation in transgenic corn transformed with an overexpression vector comprising SEQ ID NO: 3.

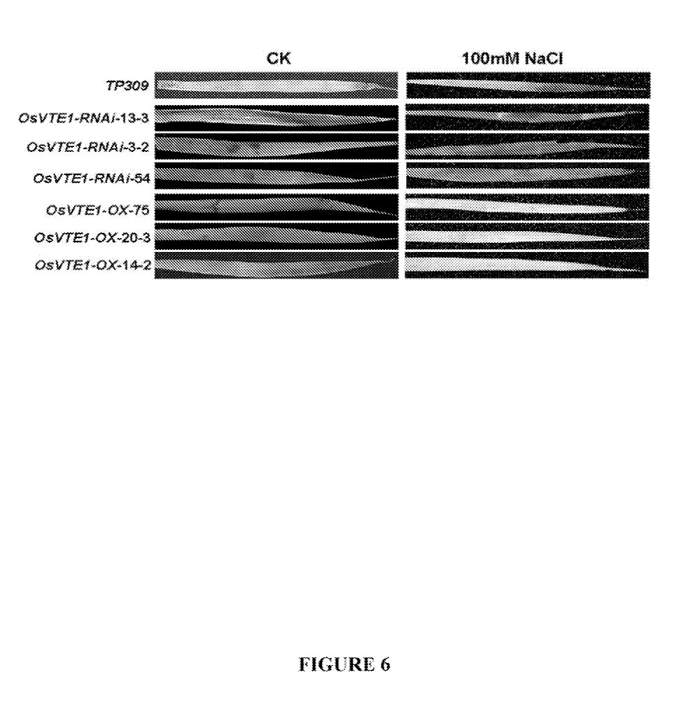

As shown in FIG. 6, salt stress produced significantly fewer brown spots in OsVTE1-OX plants than in control TP309 plants. In even starker contrast, OsVTE1-RNAi plants showed significant damage, as almost the entire leaf of all three lines were turned brown.

### Example 5

### Effect of Drought Stress on TP309 Rice

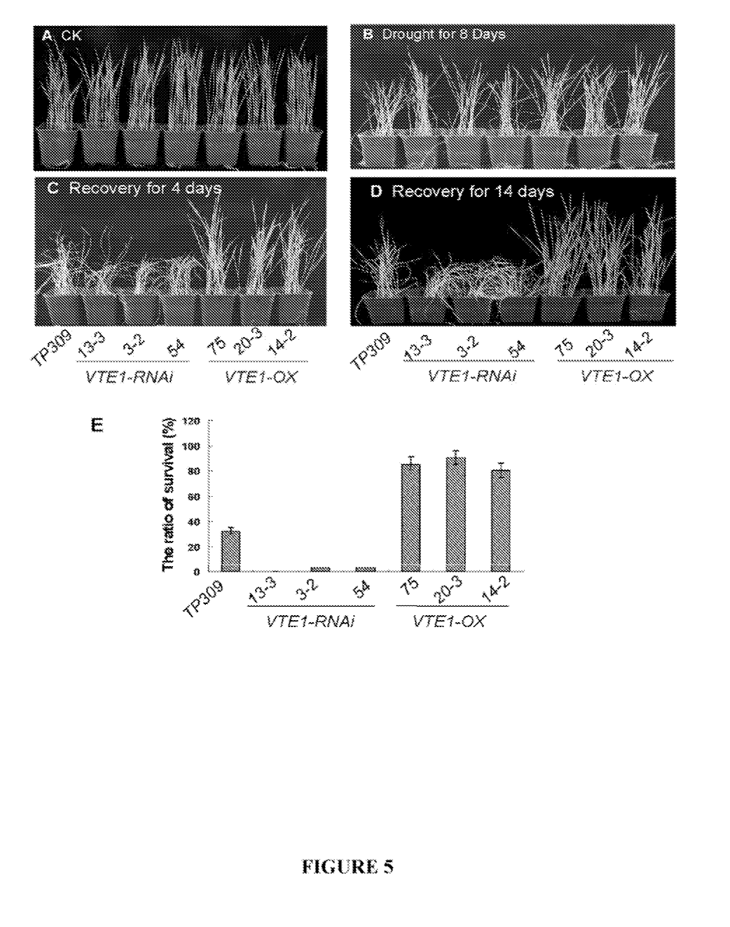

TP309 rice were transformed using both pBin438-OsTC and pZH01-OsVTE1 vectors as described in Example 2. Transgenic OsVTE1-OX, OsVTE1-RNAi, and control TP309 plants were sown in pots (8×10 cm) containing vermiculite soaked with water. All plants were grown under white fluorescent light (600 μmol/m²/s, 12 h light period/day) at 28° C. and in 75% relative humidity. After being allowed to grow for four weeks (CK; see FIG. 5A), all pots were withdrawn from water and were allowed to dry naturally. A similar protocol is used to evaluate the effect of drought stress on transgenic corn transformed with an overexpression vector comprising SEQ ID NO: 3.

During drought stress, the OsVTE1-OX plants showed a significant delay in leaf-rolling as compared to control TP309 plants. After eight days, almost all leaves of OsVTE1-RNAi and control plants were completely rolled, whereas only a small portion of the leaves in OsVTE1-OX plants had slightly rolled (FIG. 5B). All plants were then re-watered. Four days later, the leaves of the OsVTE1-OX plants were beginning to expand again, while the other plants began to wither (FIG. 5C). Two weeks after re-watering began, more than 80% of OsVTE1-OX plants recovered, but only about 40% of the TP309 controls recovered, and less than 5% of OsVTE1-RNAi plants recovered (FIGS. 5D and 5E).

### Example 6

### Tocopherol Extraction and Measurement

In plants, the composition of tocopherol differs between different species and within different tissues within the same species (summarized by Grusak and DellaPenna, 1999). To investigate whether expression of OsVTE1 affected tocopherol biosynthesis in rice, the composition of the tocopherol pool in the vegetative stage of TP309 rice was examined. Tocopherols were extracted essentially as described by Panfili et al. (2003). Plants were grown for four weeks under normal conditions. Approximately 150 mg of leaves of different plant lines were ground up and homogenized in liquid nitrogen, and then extracted with 1 mL of 100% methanol. After incubating for 30 minutes at 30° C., samples were centrifuged at 12,000 rpm for 10 minutes at 4° C. The supernatant was transferred to a new tube and the pellet was extracted again with 800 μL 100% methanol for 30 minutes at 30° C., and all supernatants were pooled after centrifuging twice. A similar protocol is used to evaluate to measure tocopherol production in transgenic corn transformed with an overexpression vector comprising SEQ ID NO: 3.

As shown in Table 1, the tocopherol pools in all plants consisted more than 70% α-tocopherol. The amount of γ-tocopherol decreased sharply in OsVTE1-RNAi plants, especially the OsVTE1-RNAi-13-3 line. In contrast, γ-tocopherol content increased significantly in OsVTE1-OX plants, especially in the OsVTE1-OX-20-3 line. Interestingly, both an OsVTE1-RNAi and an OsVTE1-OX line had statistically significant greater levels of α-tocopherol than controls.

TABLE 1

| | | | Total tocopherol |
|---|---|---|---|
| | γ-tocopherol | α-tocopherol | (γ + α) |
| Sample | (μg/g FW) | (μg/g FW) | (μg/g FW) |
| TP309 | 11.68 ± 0.69 | 247.21 ± 10.78 | 258.89 ± 11.47 |
| OsVTE1- | 0.00 ** | 272.92 ± 19.56 | 272.92 ± 19.56 |
| RNAi- | | | |
| 13-3 | | | |
| OsVTE1- | 7.09 ± 1.18 * | 276.00 ± 61.18 | 283.09 ± 62.36 |
| RNAi-3-2 | | | |
| OsVTE1- | 2.45 ± 2.13 ** | 304.96 ± 76.28 * | 307.41 ± 78.41 * |
| RNAi-54 | | | |
| OsVTE1- | 11.13 ± 1.15 | 283.66 ± 30.87 | 294.79 ± 32.02 * |
| OX-75 | | | |
| OsVTE1- | 61.55 ± 7.26 ** | 329.62 ± 61.78 ** | 452.55 ± 69.04 ** |
| OX-20-3 | | | |
| OsVTE1- | 12.40 ± 1.18 | 282.43 ± 8.97 | 294.83 ± 10.15 * |
| OX-14-2 | | | |

Data are means of three independent experiments ±SD.
* = significantly different from control plants at p < 0.01.
** = significantly different from control plants at p < 0.001.

The recovery for α- and γ-tocopherol from rice leaves was determined by resolving the methanol extracts on a PHENOMENEX® C18 reverse-phase column (150 mm length and 5 μm particle size), with a methanol:water (98:2) mobile phase at flow rate of 1 mL/min. Tocopherol levels were quantified by fluorescence detection (excitation at 290 nm and emission at 325 nm) using standards purchased from Sigma Chemicals. The recovery rates for α- and γ-tocopherol were 89.5% and 90.4% respectively.

The disclosure of every patent, patent application, and publication cited herein is hereby incorporated herein by reference in its entirety.

While this invention has been disclosed with reference to specific embodiments, it is apparent that other embodiments and variations of this invention can be devised by others skilled in the art without departing from the true spirit and scope of the invention. The appended claims include all such embodiments and equivalent variations.

1-17. (canceled)

18. A method of increasing salt tolerance in a rice plant, the method comprising expressing in the rice plant an isolated tocopherol cyclase (TC) polynucleotide selected from the group consisting of:
(a) a nucleic acid comprising a nucleotide sequence of SEQ ID NO: 1 or SEQ ID NO: 3;
(b) a nucleic acid comprising a nucleotide sequence at least 95% identical to the nucleotide sequence of SEQ ID NO: 1 or SEQ ID NO: 3;
(c) a nucleic acid that specifically hybridizes to the complement of the nucleotide sequence of SEQ ID NO: 1 or SEQ ID NO: 3 under stringent hybridization conditions comprising 50% formamide and 1 mg of heparin overnight at 40° C. and wash conditions comprising 0.2×SSC at 65° C. for 15 minutes;
(d) a nucleic acid comprising an open reading frame encoding a TC protein comprising a polypeptide sequence of SEQ ID NO: 2;
(e) a nucleic acid comprising an open reading frame encoding a TC protein comprising a polypeptide sequence at least 95% identical to SEQ ID NO: 2; and
(f) a nucleic acid comprising a nucleotide sequence that is the complement of any one of (a) to (e).

19. The method of claim 18, wherein the method comprises:
(a) introducing the isolated TC polynucleotide into a rice cell; and
(b) regenerating the rice plant expressing the isolated TC polynucleotide from the rice cell of (a).

20. The method of claim 19, wherein the method further comprises hybridizing the transgenic rice plant of (b) with a non-transgenic rice plant.

21. The method of claim 18, wherein the nucleic acid comprises an open reading frame encoding a TC protein comprising the polypeptide sequence of SEQ ID NO: 2 or a polypeptide sequence at least 95% identical to SEQ ID NO: 2.

22. The method of claim 18, wherein the nucleic acid comprises an open reading frame encoding a TC protein comprising the polypeptide sequence of SEQ ID NO: 2.

23. The method of claim 18, wherein the nucleic acid comprises the nucleotide sequence of SEQ ID NO: 1.

24. The method of claim 18, wherein the nucleic acid consists of the nucleotide sequence of SEQ ID NO: 1.

25. The method of claim 18, wherein the nucleic acid comprises the nucleotide sequence of SEQ ID NO: 3.

26. The method of claim 18, wherein the nucleic acid consists of the nucleotide sequence of SEQ ID NO: 3.

27. A method of increasing drought tolerance in a rice plant, the method comprising expressing in the rice plant an isolated tocopherol cyclase (TC) polynucleotide selected from the group consisting of:
(a) a nucleic acid comprising a nucleotide sequence of SEQ ID NO: 1 or SEQ ID NO: 3;
(b) a nucleic acid comprising a nucleotide sequence at least 95% identical to the nucleotide sequence of SEQ ID NO: 1 or SEQ ID NO: 3;
(c) a nucleic acid that specifically hybridizes to the complement of the nucleotide sequence of SEQ ID NO: 1 or SEQ ID NO: 3 under stringent hybridization conditions comprising 50% formamide and 1 mg of heparin overnight at 40° C. and wash conditions comprising 0.2×SSC at 65° C. for 15 minutes;
(d) a nucleic acid comprising an open reading frame encoding a TC protein comprising a polypeptide sequence of SEQ ID NO: 2;
(e) a nucleic acid comprising an open reading frame encoding a TC protein comprising a polypeptide sequence at least 95% identical to SEQ ID NO: 2; and
(f) a nucleic acid comprising a nucleotide sequence that is the complement of any one of (a) to (e).

28. The method of claim 26, wherein the method comprises:
(a) introducing the isolated TC polynucleotide into a rice cell; and
(b) regenerating the rice plant expressing the isolated TC polynucleotide from the rice cell of (a).

29. The method of claim 28, wherein the method further comprises hybridizing the transgenic rice plant of (b) with a non-transgenic rice plant.

30. The method of claim 27, wherein the nucleic acid comprises an open reading frame encoding a TC protein comprising the polypeptide sequence of SEQ ID NO: 2 or a polypeptide sequence at least 95% identical to SEQ ID NO: 2.

31. The method of claim 27, wherein the nucleic acid comprises an open reading frame encoding a TC protein comprising the polypeptide sequence of SEQ ID NO: 2.

32. The method of claim 27, wherein the nucleic acid comprises the nucleotide sequence of SEQ ID NO: 1.

33. The method of claim 27, wherein the nucleic acid consists of the nucleotide sequence of SEQ ID NO: 1.

34. The method of claim 27, wherein the nucleic acid comprises the nucleotide sequence of SEQ ID NO: 3.

35. The method of claim 27, wherein the nucleic acid consists of the nucleotide sequence of SEQ ID NO: 3.
